(19) United States
(12) Patent Application Publication
(10) Pub. No.: US 2012/0101281 A1
(43) Pub. Date: Apr. 26, 2012

(54) PROCESS FOR THE PREPARATION OF DESLORATADINE

(75) Inventors: Balaguru Murugesan, Tamil Nadu (IN); Swargam Sathyanarayana, Andhra Pradesh (IN); Mohan Prasad, Haryana (IN)

(73) Assignee: RANBAXY LABORATORIES LIMITED, New Delhi, Delhi (IN)

(21) Appl. No.: 13/258,661

(22) PCT Filed: Mar. 26, 2010
(86) PCT No.: PCT/IB2010/051341

§ 371 Date: Dec. 8, 2011

(30) Foreign Application Priority Data
Mar. 26, 2009 (IN) 595/DEL/2009

(51) Int. Cl. C07D 221/16

(52) U.S. Cl. 546/93

(57) ABSTRACT

The present invention provides a process for the preparation of desloratadine comprising contacting loratadine with a mixture of a weak inorganic base and sodium or potassium hydroxide in a ratio, ranging from 0.01 to 0.15 equivalents of sodium or potassium hydroxide per equivalent of weak inorganic base, in one or more suitable solvents) followed by isolation.

### FIELD OF THE INVENTION

The present invention provides a process for the preparation of desloratadine.

### BACKGROUND OF THE INVENTION

Desloratadine is chemically described as 8-chloro-6,11-dihydro-11-(4-piperidinylidene)-5H-benzo[5,6]cyclohepta[1,2-b] pyridine and is represented by Formula I.

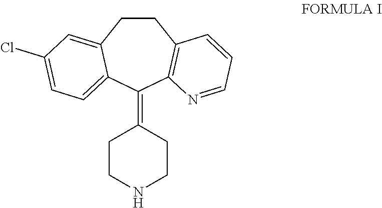

Desloratadine is a metabolite of loratadine, having non-sedative antihistaminic activity and is known from U.S. Pat. No. 4,659,716. Several methods for the preparation of desloratadine are known in the literature, such as those described in U.S. Pat. Nos. 4,659,716; 5,719,148; WO 03/086275 and WO 2004/029039, which are incorporated herein by reference.

The processes described in the literature for the preparation of desloratadine are not suitable for an industrial scale preparation as they either involve the use of poisonous cyanogen bromide reagent; thereby producing poisonous methyl bromide or requiring further purification by recrystallization.

WO 2004/029039 describes a process for the preparation of desloratadine by decarboethoxylation of loratadine with sodium hydroxide or potassium hydroxide in neat alcohol. It also discloses that inorganic bases, such as lithium compounds, and carbonates of alkali metals do not work.

### SUMMARY OF THE INVENTION

In one general aspect, the present invention provides for a process for the preparation of desloratadine of Formula I,

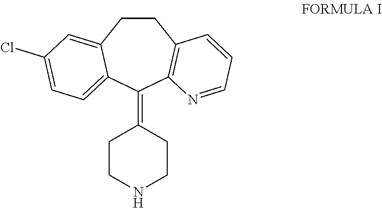

whereby the process includes contacting loratadine with a mixture of a weak inorganic base and sodium or potassium hydroxide in a ratio, ranging from 0.01 to 0.15 equivalents of sodium or potassium hydroxide per equivalent of weak inorganic base, in one or more suitable solvent(s) followed by isolation.

Embodiments of the process may include one or more of the following features. For example, the weak inorganic base may include lithium hydroxide monohydrate, lithium carbonate, sodium carbonate, potassium carbonate and sodium bicarbonate.

The suitable solvent includes water, alcohols, hydrocarbons, chlorinated hydrocarbons, ethers, alkyl acetates, ketones, dipolar aprotic solvents and mixtures thereof. The suitable alcohols may include straight and branched chain alcohols, aromatic alcohols or polyols; and mixtures thereof with water. For example, the suitable solvent is a mixture of ethanol and water.

The reaction of loratadine with a mixture of a weak inorganic base and sodium or potassium hydroxide is carried out at a temperature range of from about ambient temperature to about reflux temperature of the suitable solvent(s). The reaction is refluxed for about 20 hours to about 60 hours.

In another general aspect there is provided desloratadine comprising less than 0.1% w/w of a compound of Formula II.

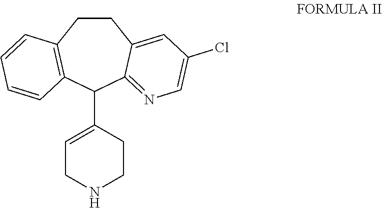

### DETAILED DESCRIPTION OF THE INVENTION

Loratadine, which is used as a starting material for the preparation of desloratadine, may be obtained by any of the processes known in the literature, such as those described in U.S. Pat. Nos. 4,282,233, 6,271,378, 6,084,100, WO 2004/080997, which are herein incorporated for reference only. The loratadine used as starting material may be obtained as a solution directly from a reaction mixture in which loratadine is formed, and may be used as such without isolation.

The term “contacting” includes dissolving, slurrying, stiffing, or a combination thereof.

A suitable weak inorganic base may include lithium hydroxide monohydrate, lithium carbonate, sodium carbonate, potassium carbonate, sodium bicarbonate and the like. Preferably, lithium hydroxide monohydrate may be used for decarboethoxylation.

The mixture of weak inorganic base and sodium or potassium hydroxide may be added in a ratio of about 0.01 to about 0.15 equivalent of sodium or potassium hydroxide per equivalent of weak inorganic base.

The suitable solvent(s) may include water, alcohols, hydrocarbons, chlorinated hydrocarbons, ethers, alkyl acetates, ketones, dipolar aprotic solvents; and/or mixtures thereof. Examples of alcohols include straight and branched chain alcohols, such as, methanol, ethanol, n-propanol, iso-propanol, and the like, cyclic alcohols, such as, cyclopentanol, cyclohexanol, and the like, aromatic alcohols, such as, substituted or un-substituted benzyl alcohols, polyols, such as, polyethylene glycol, and the like. Examples of hydrocarbons include hexane, cyclohexane, benzene, toluene, and the like. Examples of chlorinated hydrocarbons include chloroform, dichloromethane, and the like. Examples of ethers include diethyl ether, diisopropyl ether, tetrahydrofuran, and the like. Examples of alkyl acetates include ethyl acetate, iso-propyl acetate, and the like. Examples of ketones include acetone, methyl ethyl ketone, methyl isobutyl ketone, and the like. Examples of dipolar aprotic solvents include acetonitrile, dimethylformamide, dimethylsulphoxide, and the like. Preferably, a mixture of ethanol and water may be used.

The reaction may be carried out at ambient temperature to reflux temperature of the suitable solvent(s). The reaction mixture comprising loratadine and a weak inorganic base in suitable solvent(s) may be refluxed for about 20 hours to about 60 hours, preferably, for about 34 hours to about 40 hours.

The reaction mixture may be cooled to a temperature of about 20° C. to about 60° C., preferably, to about 40° C. to about 45° C. The cooling may be carried out in a period of about 10 minutes to about 60 minutes, preferably for about 30 minutes to about 40 minutes.

Water may be added to the reaction mixture. The suitable solvent(s) may be recovered under reduced pressure.

The contents may be cooled to a temperature of about 0° C. to about 25° C., preferably to about 10° C. to about 15° C.

The reaction mixture may be extracted with a suitable solvent that may include alkyl acetates, ethers, hydrocarbons, chlorinated hydrocarbons, dipolar aprotic solvents; and/or mixtures thereof. Examples of alkyl acetates include ethyl acetate, iso-propyl acetate; and the like. Examples of ethers include diethyl ether, diisopropyl ether, tetrahydrofuran; and the like. Examples of hydrocarbons include hexane, cyclohexane, benzene, toluene; and the like. Examples of chlorinated hydrocarbons include chloroform, dichloromethane; and the like. Examples of dipolar aprotic solvents include acetonitrile, dimethylformamide, dimethylsulphoxide; and the like. Preferably, the reaction mixture may be extracted with ethyl acetate.

The reaction mixture may be filtered and the layers may be separated. The organic layer may be treated with activated carbon and filtered. Solvent(s) may be recovered under atmospheric pressure at a temperature of about 60° C. to about 90° C. Preferably, the solvent(s) may be recovered at a temperature of about 80° C. to about 90° C.

Isolation may be accomplished by concentration, precipitation, cooling, filtration or centrifugation, or a combination thereof followed by drying. Preferably, isolation is accomplished by adding a suitable solvent that includes ketones, alcohols, chlorinated hydrocarbons, ethers, acetonitrile, N, N-dimethylformamide; and the like. Examples of ketones include acetone, methyl ethyl ketone, methyl isobutyl ketone; and the like. Preferably, desloratadine may be isolated by adding acetone.

Desloratadine, obtained by the process of the invention, is free of the undesired isomer of Formula II. The term “free of undesired isomer of Formula II”, refers to desloratadine having less than 0.1% w/w of undesired isomer of Formula II, as determined by HPLC.

### HPLC Method

### Column: Prodigy ODS-3,5 μm (250×4.6 mm)

### Column Oven Temperature: 300° C.

### Gradient: Buffer (pH 4.5), Methanol and THF

### Detector: 270 nm

In the foregoing section, embodiments are described by way of example to illustrate the process of invention. However, this is not intended in any way to limit the scope of the present invention. Several variants of the example would be evident to persons ordinarily skilled in the art.

### Example: Preparation of Desloratadine

Ethane (600 mL) was added to a clear solution containing sodium hydroxide (11 g) in de-ionized water (400 mL) at a temperature of about 30° C. to 32° C. Lithium hydroxide monohydrate (131 g) was added. The contents were stirred for about 5 to 10 minutes to obtain a uniform mixture. Loratadine (100 g) was added to the mixture at a temperature of about 35° C. to 45° C. The temperature of the reaction mixture was raised to reflux temperature. The reaction mixture was stirred for about 34 to 40 hours. The reaction mixture was slowly cooled to about 40° C. to 45° C. in about 30 minutes to 40 minutes. De-ionized water (660 mL) was added. Ethanol was completely recovered under reduced pressure. The contents were cooled to about 10° C. to 15° C. in about 30 to 40 minutes. Ethyl acetate (500 mL) was added. The contents were stirred for about 15 to 20 minutes, filtered through celite and the bed was washed with ethyl acetate. The layers were separated and ethyl acetate (200 mL) was added to the aqueous layer. The contents were stirred for about 15 to 20 minutes at ambient temperature and the layers were separated. 10% aqueous sodium chloride solution (400 mL) was added to the combined organic layers; this was stirred for 5 to 10 minutes and the layers were separated. The organic layer was treated with activated carbon, stirred for about 60 minutes, filtered and washed with ethyl acetate. Organic layers were combined and ethyl acetate was recovered completely at a temperature of about 80° C. to 90° C. under atmospheric pressure. The residue was cooled to about 20° C. to 25° C. and acetone (200 mL) was added. The contents were stirred for about 15 hours, filtered, washed with chilled acetone at a temperature of about 0° C. to 5° C. and dried. De-ionized water (300 mL) was added to the wet cake. The contents were stirred for about 2 hours, filtered and dried in an air oven to obtain desloratadine.

### Yield: 60.9 g

### Total Related Substances by HPLC: 0.266% w/w

Isomer of Formula II: 0.051% w/w

We claim:

1. A process for the preparation of desloratadine of Formula I 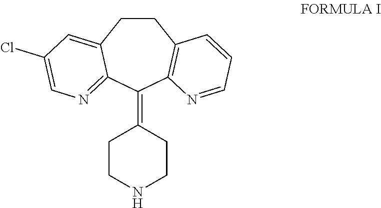
comprising contacting loratadine with a mixture of a weak inorganic base and sodium or potassium hydroxide in a ratio, ranging from 0.01 to 0.15 equivalents of sodium or potassium hydroxide per equivalent of weak inorganic base, in one or more suitable solvent(s) followed by isolation.

2. The process according to claim 1, wherein the weak inorganic base comprises lithium hydroxide monohydrate, lithium carbonate, sodium carbonate, potassium carbonate and sodium bicarbonate.

3. The process according to claim 1, wherein the suitable solvent comprises water, alcohols, hydrocarbons, chlorinated hydrocarbons, ethers, alkyl acetates, ketones, dipolar aprotic solvents and mixtures thereof.

4. The process according to claim 3, wherein the alcohols comprises straight and branched chain alcohols, aromatic alcohols or polyols; and mixtures thereof with water.

5. The process according to claim 1, wherein the suitable solvent comprises a mixture of ethanol and water.

6. The process according to claim 1, wherein the reaction of loratadine with a mixture of a weak inorganic base and sodium or potassium hydroxide is carried out at a temperature range of from about ambient temperature to about reflux temperature of the suitable solvent(s).

7. The process according to claim 6, wherein the reaction is refluxed for about 20 hours to about 60 hours.

8. Desloratadine comprising less than 0.1% w/w of a compound of Formula II. [chemical structure]
